(19) United States
(12) Patent Application Publication
(10) Pub. No.: US 2022/0073905 A1
(43) Pub. Date: Mar. 10, 2022

(54) METHODS AND MATERIALS FOR SINGLE CELL TRANSCRIPTOME-BASED DEVELOPMENT OF AAV VECTORS AND PROMOTERS

(71) Applicant: University of Pittsburgh - Of the Commonwealth System of Higher Education, Pittsburgh, PA (US)

(72) Inventors: Leah Byrne, Pittsburgh, PA (US); William R. Stauffer, Pittsburgh, PA (US)

(21) Appl. No.: 17/416,601

(22) PCT Filed: Dec. 4, 2019
(86) PCT No.: PCT/US2019/068489

§ 371 Date: Jun. 21, 2021

Related U.S. Application Data
(60) Provisional application No. 62/785,818, filed on Dec. 28, 2018.

(51) Int. Cl. C12N 15/10; C12N 15/86

(52) U.S. Cl. CPC C12N 15/1065; C12N 2750/14143; C12N 15/86; C12N 15/1096

(57) ABSTRACT

This document provides a high throughput method for the creation of AAV vectors and/or promoter sequences with high efficiency and/or specificity for multiple cell types.

### CROSS-REFERENCE To RELATED APPLICATIONS

This application claims the benefit of U.S. Patent Application Ser. No. 62/785,818, filed on Dec. 28, 2018. The disclosure of the prior application is considered part of (and is incorporated by reference in) the disclosure of this application.

### STATEMENT AS To FEDERALLY SPONSORED RESEARCH

This invention was made with government support under MH113095 awarded by National Institutes of Health. The government has certain rights in the invention.

### BACKGROUND

### 1. Technical Field

This document relates to methods and materials for single cell transcriptome-based development of adeno-associated virus (AAV) vectors and promoters. For example, this document provides efficient and high-throughput methods for creating effective AAV vectors.

### 2. Background Information

Efficient and targeted gene delivery is fundamental to the success of gene therapies and circuit-based tools such as optogenetics. Sufficient levels of gene expression in the desired cell type is essential and off-target expression ideally should be minimized for precisely targeted therapies, patient safety, and circuit specific manipulation.

An attractive vector system for gene therapy is based on genetically engineered and modified adeno-associated viruses (AAVs). Existing screening methodologies for creating AAVs, however, require large numbers of cells, and focus on only one cell type at a time employing many rounds of selection. These also are based on DNA screening. Thus, there is a need for a more efficient and high-throughput method for the creation of AAV vectors for gene therapy.

### SUMMARY

This document provides high throughput methods for the creation of AAV vectors and promoter sequences with high efficiency and/or specificity for multiple cell types. In some embodiments, first, libraries of AAV mutants containing multiple variants (between approximately 20, or approximately 50, or approximately 100, or approximately 1000 or approximately 10,000, or approximately 10,000, or approximately 1,000,000 and up to approximately 10⁶ or approximately 10⁷ variants) are created, and injected or otherwise introduced into tissues (e.g., retina, brain, muscle, etc.) of animals, typically primates. In some cases, the AAV libraries provided herein can be injected directly into desired tissue (e.g., an intravitreal injection) or can be injected systemically.

Injection (or introduction or infection/transfection otherwise) into certain tissue in culture, such as retinal organoids, also can be used instead of in vivo screening. These libraries are created such that each AAV variant in the library contains a unique “DNA barcode” (i.e., unique DNA sequences, that are part of the AAV genome, and that indicate the identity of a viral variant), which allows for tracking of either an AAV capsid or a synthetic upstream promoter.

Secondly, in some embodiments, after injection, the AAV vectors compete with each other in vivo (or in tissue in culture), such that stronger AAV vectors or promoters lead to greater expression levels of the DNA barcodes, and more specific AAV vectors or promoters lead to increased levels of expression in one or more cell types relative to all other cell types. Thereafter, single cell or single nucleus microfluidics technology (from companies such as 10X GENOMICS or DOLOMITE BIO) is used to create cDNA libraries of individual cells (or nuclei).

Analysis then is performed to identify optimal vectors, according to specificity, expression level, and/or other desirable characteristics, based on the presence and quantity of DNA barcodes in transcriptomes from many different cell types in parallel. Selection can be performed on two levels: (a) highly diverse viral capsid libraries can be screened for vectors with efficient and specific tropism, and (b) enhancer/promoter constructs can be evaluated for their ability to drive expression in specific cell populations.

In some cases, the performance of viral capsids is evaluated on the basis of mRNA transcription levels rather than DNA. RNA can be used as an indicator of virus function because it reflects the ability of vectors to drive expression of the protein payload, rather than merely enter a cell. Also, as described herein, the methods provided herein can involve using multiple cell types, typically, though not exclusively, in vivo, which is an improvement over typical methods involving bulk tissue or one cell type at a time employing multiple rounds of selection.

In some cases, the methods provided herein can be performed in many tissues, including the primate retina, brain, muscle, or other tissue, to maximize the translational potential of resulting vectors. In some cases, a high throughput screening approach provided herein can allow for the identification and characterization of viral variants and promoters with desired properties, including broad tropism and specificity.

In general, one aspect of this document features a method comprising (a) creating a library of AAV mutants or promoters, wherein each AAV within the library comprises a unique DNA barcode, or each promoter construct comprises a unique DNA barcode, (b) packaging of AAV mutants or promoters with a double (for capsid libraries) or triple (for some capsid or promoter libraries) transfection protocol into a packaging cell line, (c) delivering the library of AAV mutants into one or more tissues of an animal host, or infecting tissue in culture, (d) maintaining the library of AAV mutants in vivo or culturing the library of viruses in tissue in culture for a period of time suitable for the AAV vectors within the library of AAV mutants to compete with each other within the one or more tissues of an animal host or cultured tissue into which the library of AAV mutants has been delivered, and (e) employing single cell or single nucleus microfluidics methodologies to create single cell or single nucleus cDNA libraries from cells within the one or more tissues of the animal host into which the library of AAV mutants has been delivered. The step (e) can employ single cell microfluidics technology. The step (e) can employ single nucleus microfluidics technology. The one or more tissues of an animal host can comprise neural tissue. The neural tissue can comprise central neural system tissue. The central nervous system tissue can be brain tissue. The neural tissue can comprise peripheral nervous system tissue. The one or more tissues of an animal host can comprise retinal tissue. The one or more tissues of an animal host can comprise muscle tissue. The muscle tissue can comprise striated muscle. The muscle tissue can comprise cardiac muscle. The muscle tissue can comprise smooth muscle. The animal host can be a primate. The primate can be an Old World monkey. The Old World monkey can be a Rhesus macaque (Macaca mulatta). The primate can be an ape of the family Hylobatidae or Hominidae. The primate can be a primate that is not of the genus Homo. The primate can be a primate that is not of the genus Pan. The delivery of the library of AAV mutants can be via injection into the tissue.

In another aspect, this document features a method for obtaining an AAV mutant having the ability to infect a desired cell type in vivo and be maintained in vivo within the cell type for at least one week. The method comprises (or consists essentially of or consists of) (a) introducing a library of AAV mutants into an animal host comprising the cell type, wherein each AAV within the library comprises a unique DNA barcode, and (b) identifying one or more AAV mutants, based on the barcode for the one or more AAV mutants, as being present in a cell of the cell type, wherein the cell was within the animal host for at least one week after the library was introduced into the animal host. The cell type can be a central nervous system cell type or peripheral nervous system cell type. The cell type can be a retinal cell type, a striated muscle cell type, a cardiac muscle cell type, or a smooth muscle cell type. The animal host can be a primate. The primate can be an Old World monkey. The primate can be a Rhesus macaque (Macaca mulatta). The primate can be an ape of the family Hylobatidae or Hominidae. The primate can be a primate that is not of the genus Homo. The primate can be a primate that is not of the genus Pan. The library can be introduced into the animal host via injection into tissue comprising the cell type. The at least one week can be from one week to 12 weeks.

In another aspect, this document features a method for obtaining a promotor sequence from a library of AAV viruses. The method comprises (or consists essentially of or consists of) (a) introducing the library into an animal host comprising a cell type, wherein each AAV within the library comprises a unique promotor sequence configured to drive expression of a fluorescent polypeptide, and (b) identifying one or more promotor sequences, based on the expression of the fluorescent polypeptide, as being present in a cell of the cell type, wherein the cell was within the animal host for at least one week after the library was introduced into the animal host. The cell type can be a central nervous system cell type or peripheral nervous system cell type. The cell type can be a retinal cell type, a striated muscle cell type, a cardiac muscle cell type, or a smooth muscle cell type. The animal host can be a primate. The primate can be an Old World monkey. The primate can be a Rhesus macaque (Macaca mulatta). The primate can be an ape of the family Hylobatidae or Hominidae. The primate can be a primate that is not of the genus Homo. The primate can be a primate that is not of the genus Pan. The library can be introduced into the animal host via injection into tissue comprising the cell type. The at least one week can be from one week to 12 weeks.

In another aspect, this document features an isolated nucleic acid comprising (or consisting essentially of or consisting of) nucleic acid encoding an AAV rep polypeptide, nucleic acid encoding an AAV cap polypeptide, and a nucleic acid cassette, wherein the nucleic acid cassette comprises a promotor sequence, nucleic acid encoding a peptide tag, a nucleic acid barcode, and a polyA tail sequence. The nucleic acid encoding the AAV rep polypeptide, the nucleic acid encoding the AAV cap polypeptide, and the nucleic acid cassette can be located between two inverted terminal repeats. The nucleic acid barcode can be between 20 and 30 nucleotides in length. The isolated nucleic acid can be a plasmid.

In another aspect, this document features an isolated nucleic acid comprising (or consisting essentially of or consisting of) nucleic acid encoding an AAV cap polypeptide and a nucleic acid cassette, wherein the nucleic acid cassette comprises a promotor sequence, nucleic acid encoding a fluorescent polypeptide, and a polyA tail sequence, and wherein the isolated nucleic acid lacks nucleic acid encoding a full length rep polypeptide. The nucleic acid encoding the AAV cap polypeptide and the nucleic acid cassette can be located between two inverted terminal repeats. The isolated nucleic acid can comprise nucleic acid encoding a rep polypeptide amino acid sequence that is no more than 25 percent, no more than 50 percent, no more than 75 percent, or no more than 85 percent of the amino acid sequence of a full length rep polypeptide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts the strategy and maps of packaging constructs for single-cell AAV capsid and promoter library screening, according to some embodiments.

FIG. 2 depicts a method involving single-cell screening of AAV capsids and promoters, according to some embodiments. The method is illustrated in retinal tissue as an example. Panel A: Libraries of barcoded AAVs are injected into tissue. Virus variants from the library infect different cells with different efficiencies. Panel B: Efficient viruses enter cells, traffic to the nucleus, and lead to expression of mRNA. Panel C: Tissue is dissociated into single cells, and mRNA from individual cells is tagged with cell-specific DNA barcodes. Panel D: The transcriptome profile of individual cells is analyzed to determine cell type, as well as which AAVs have infected the cell, and AAV specificity and efficiency. Panel E: For enhancer/promoter libraries, the libraries are packaged in a single AAV capsid with broad tropism. Panel F: Different promoters drive varying levels of gene expression in individual cell types. Panel G: Single cell suspensions are created and mRNA from individual cells is tagged with cell-specific DNA barcodes. Panel H: The transcriptome profile of individual cells is analyzed to identify cell types and determine promoter specificity and efficiency.

FIG. 3 depicts an example of library construction of AAV serotypes screened in vivo in primate retina and brain. A library of 23 AAVs were packaged individually with a genome containing a ubiquitous promoter driving expression of a green fluorescent peptide (GFP) fused to a unique DNA.

FIG. 4 depicts GFP expression in primate retina and brain following injection of the AAV libraries as described in the description of FIG. 3. These variants were packaged, pooled, and injected into Rhesus macaque retina, pre-frontal cortex (PFC), and striatum. Injection of the library resulted in GFP expression in retina and brain.

FIG. 5 displays data concerning the identification of intrinsically photosensitive retinal ganglion cells (RGCs) in Rhesus retina, and recovery of barcodes from specific AAV serotypes. Each circle is an individual cell. Panel A shows that OPN4^{+/-} cells cluster in ICA space. Panel B represents the identification of OPN4^{+/-} and POU4F2^{+/-} RGC cells. Larger circles indicate cells from which AAV genomes (identified by their barcodes) were recovered.

FIG. 6 is a heat map of AAV tropisms across cell types. Quantification of 23 existing serotypes reveals that AAVs evolved for infectivity in retina (K916, K94, 7m8, K912, NHP26) outperform other variants, as expected. In contrast, AAV9 based vectors are the top performing variants in putamen, validating the approach. AAV variants that did not infect the analyzed cell types are not shown in the heat map.

FIG. 7A is a map of AAV vectors of a library provided herein with no intervening sequence (IVS) between the minimal promoter and small peptide sequence. FIG. 7B is a map of AAV vectors of a library provided herein with IVS between the minimal promoter and small peptide sequence.

FIG. 8 is a map of AAV vectors of a library that contain AAV promoters (P40 or P19+P40) to drive cap expression, and then a promoter (e.g., a CAG promotor) to drive a transgene (e.g., a CAG-GFP, a CAG-GFP11, or a CAG-split GFP with a membrane signaling peptide) inside the ITRs.

FIG. 9 is a map of a rep in trans plasmid that contains the full rep sequence with no ITRs.

FIG. 10 contains graphs of clusters created from marmoset macula scATAC-seq data (using SnapATAC) and then integrated with scRNA-seq data from the same sample. Cell types were predicted based on gene expression from the integrated scRNA-seq data (using Seurat). RHO=rods specific gene; RGR=Muller Glia specific gene; and GRIK1=Off bipolar cell specific gene.

FIGS. 11A-C contain disease gene profiles (top left=rhesus macaque macula; bottom left=marmoset macula; top right=marmoset superior; bottom right=marmoset inferior) determined for RS1 (Retinoschisin), USH2A (Usherin), and ABCA4 (ATP binding cassette subfamily A member 4), respectively.

### DETAILED DESCRIPTION

This document provides methods that comprise (a) creating a library of AAV mutants, wherein each AAV within the library comprises a unique DNA sequence (barcode) that can be tracked to evaluate virus performance, (b) packaging of AAV variants or promoters with a double (for capsid libraries) or triple (for some capsid and promoter libraries) transfection protocol into a packaging cell line, (c) injecting or otherwise introducing the library of AAV mutants into one or more tissues of an animal host or cultured tissue, (d) permitting a sufficient period of time for the library of AAV mutants for the AAV vectors within the library of AAV mutants to compete with each other in vivo (or within cultured tissue) and infect the one or more tissues of the animal host into which the library of AAV mutants has been injected or otherwise introduced, (e) employing single cell or single or nucleus microfluidics methodologies to create single cell or single or nucleus cDNA libraries from cells within the one or more tissues of the animal host into which the library of AAV mutants has been injected.

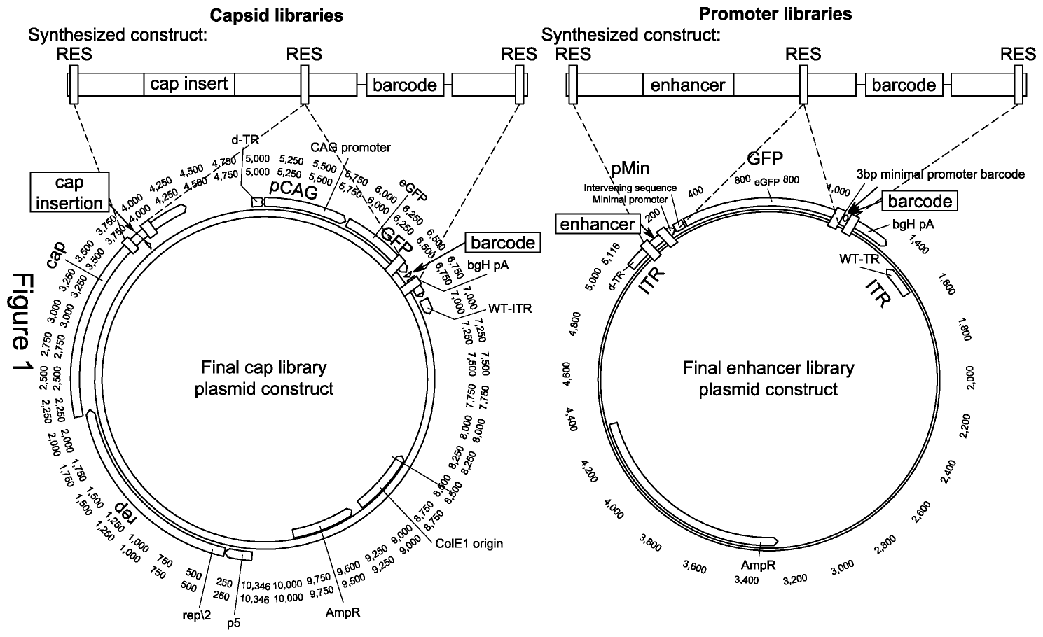

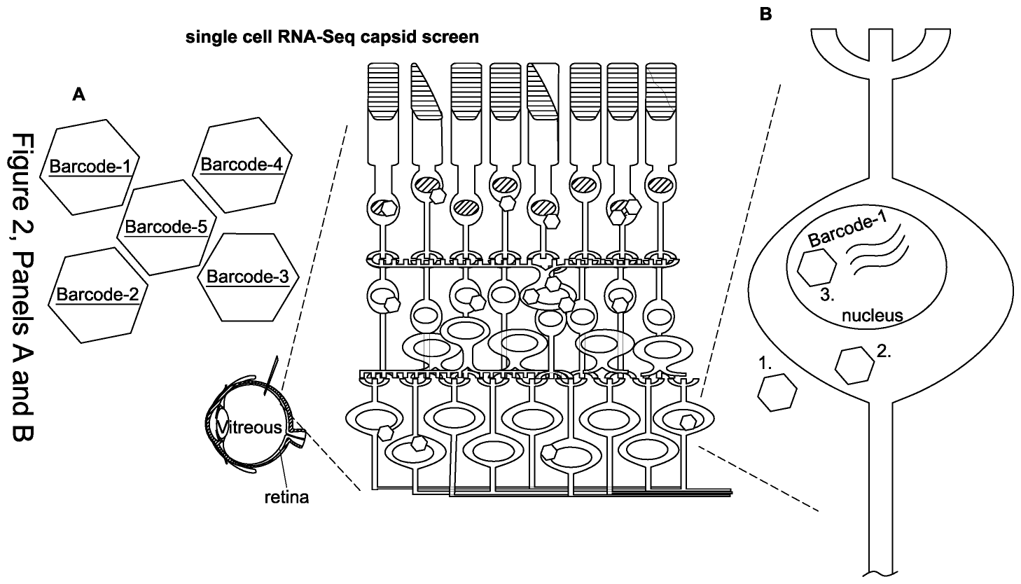

In an embodiment, the library employed in a method provided herein is a (i.e., one or more) highly complex library of AAV mutants. One map and cloning plan for making highly complex libraries is shown in FIG. 1.

As such, wherein the library involves variant AAV capsids, the method provided herein can afford a high-throughput method of creating AAV vectors with high efficiency and/or specificity for infection of targeted or multiple cell types. Similarly, wherein the library involves AAV with variant up-stream promoters operably linked to a gene-encoding sequence, the method provided herein can afford a high-throughput method of creating AAV vectors with high efficiency and/or specificity for gene-expression of targeted or multiple cell types.

The library of AAV mutants can be constructed such that each AAV within the library of AAV mutants has a unique DNA barcode, for example as illustrated in FIG. 1. In some cases, either Cap genes or promoters/enhancers, as well as barcodes, are synthesized, cloned into a backbone, and then additional sequence is cloned between the cap gene and barcode, to complete the packaging plasmid. The barcodes are cloned into the sample plasmid as are the same plasmid as the cap genes. Pairing between AAV variants and barcodes, or promoters and barcodes, can be designed in silico and subsequently synthesized. Each AAV variant or promoter can be represented by one or more unique barcodes. These synthesized constructs are then cloned into AAV packaging backbones. For AAV capsid libraries, backbones can contain rep and cap sequences, (not contained within ITR sequences) so that rep and cap genes are not packaged into the AAV capsid. On the same plasmid, an AAV genome containing a promoter driving expression of a transgene, such as nucleic acid encoding GFP or another fluorescent polypeptide, fused to a unique DNA barcode, can be placed between ITR packaging signals. Thus, a single plasmid can contain the genes for producing the AAV capsid, and a viral genome containing a unique DNA sequence tag (a barcode) that can be tracked in order to evaluate viral tropism and infectivity. Viruses can then be packaged using, for example, a double transfection method, in which two plasmids: (1) rep/cap/transgene-barcode and (2) helper plasmid providing adenovirus helper functions, are transfected into packaging cells. In some cases, a triple transfection method can be used in which three plasmids ((1) a rep plasmid, (2) promoter/cap/transgene-barcode plasmid, and (3) a helper plasmid providing adenovirus helper functions) are transfected into packaging cells. For capsid libraries, which can be based on any serotype of AAV, the naturally occurring parental serotype is also included in the library as a baseline against which the efficiency and specificity of AAV variants can be measured. For promoter libraries, the library construct can contain unique promoters driving expression of a transgene fused to a unique DNA sequence (barcode) by which the strength and specificity of the promoter can be evaluated. In the case of the promoter libraries, the rep/cap genes can be provided in trans on another plasmid. In some cases, AAVs can be packaged using a triple transfection method, in which three plasmids are transfected into a packaging cell line: (1) rep/cap plasmid, (2) promoter library-barcode construct, and (3) helper plasmid. For promoter libraries, ubiquitous CAG and CMV promoters can be synthesized and used as a baseline against which the efficiency and specificity of promoters can be measured.

Once constructed, a library of AAV mutants provided herein can be packaged into a packaging cell line. For example, AAV variants or promoters can be packaged with a double (for capsid libraries) or triple (for some capsid and promoter libraries) transfection protocol. Any appropriate packaging cell line can be used including, without limitation, HEK-293 cells, HEK293T cells, and AAV 293 cells.

Once constructed, the library of AAV mutants can be injected or otherwise introduced into one or more tissues of an animal host or in vitro cultured tissue/organoids. An animal host can be any desired species of animal that AAV vectors might infect such as a primate. Examples of primates that be used as an animal host as described herein include, without limitation, New World monkeys, Old World monkeys (e.g., a Rhesus macaque (Macaca mulatta)), great apes, and lesser apes (e.g., of the family Hylobatidae (gibbons) or Hominidae (bonobos, chimpanzees, humans, gorillas, orangutans)). In some cases, the host animal is not of the genus Homo (Homo sapiens sapiens) or Pan.

The tissue into which a library of AAV mutants provided herein is injected or otherwise introduced can be any desired tissue, such as central neural system (CNS) tissue (e.g., brain and spinal cord), peripheral nervous system tissue, retinal tissue, and muscle tissue of any type (e.g., striated, cardiac, smooth muscular tissue, etc.). Of course, other tissue/organs can suitably be injected with a library of AAV mutants provided herein, such as any internal or external tissues or organs (e.g., adrenal glands, bladder, colon, esophagus, exterior barrier tissues (skin, subdermal tissue, mucus-generating tissue, etc.), kidney, liver, lungs, ovary, pancreas, rectum, small intestine, spleen, stomach, testes, thymus, ureter, among others). In some cases, tissue grown in culture, such as retinal organoids, can be used as described herein.

In some cases, a method provided herein can include permitting a sufficient period of time for the AAV vectors within the library of AAV mutants to compete with each other and infect the one or more tissues of an animal host (or within cultured tissue) into which the library of AAV mutants has been injected or otherwise introduced. This period of time will vary depending on the tissue and species of the host animal or source of tissue in vitro. In some cases, the period of time can be between about 1 week and about 12 weeks in living organisms and about 1-14 days in cultured tissue. For example, after injecting or otherwise introducing a library of AAV mutants into a living animal host, the living animal host can be maintained for 1 to 12 weeks (e.g., 1 to 8 weeks, 1 to 5 weeks, 1 to 3 weeks, 3 to 10 weeks, 5 to 10 weeks, or 3 to 6 weeks) prior to being analyzed.

Thereafter, analysis can be performed to identify optimal vectors, according to, for example, specificity, expression level, and/or other desirable characteristics (such as, but not limited to, increased infectivity, increased specificity for one or more cell types, decreased immune response, etc.), based on the presence and quantity of DNA barcodes in transcriptomes from many different cell types in parallel. In some cases, the efficiency of a virus can be determined based on the number of GFP barcodes recovered from cells of a particular type. In some cases, vectors can then be ranked according to the level of transgene expression. The virus variants with the greatest level of transgene expression compared to the most closely related parental serotype for a particular cell type can be designated as the most efficient virus for that cell type (top performers). The virus variants with the greatest level of transgene expression compared to the most closely related parental serotype for a particular cell type with lowest levels of expression in all other cells types can be designated as the most specific viruses for that cell type (top performers).

Selection can be performed on two levels: (1) highly diverse viral capsid libraries can be screened for vectors with efficient and specific tropism, and (2) enhancer/promoter constructs can be evaluated for their ability to drive expression in specific cell populations. In this context, “efficient” refers to greater infectivity of one or more cell types of one virus when compared with a second virus. Further, in this context, “specific” refers to greater infectivity of one or more cell types with decreased infectivity or expression of all other cell types of one virus when compared with a second virus. In some cases, the performance of viral capsids can be evaluated on the basis of mRNA transcription levels rather than DNA, reflecting the ability of vectors to drive expression of the protein payload, rather than merely enter a cell. These steps can be performed in any species, including the primate retina, brain, or other tissue, to maximize the translational potential of resulting vectors. In some cases, the high throughput screening approaches provided herein can allow for the identification and characterization of viral variants and promoters with desired properties, including broad tropism and specificity.

The invention will be further described in the following examples, which do not limit the scope of the invention described in the claims.

### EXAMPLES

### Example 1—Methods for Single Cell Transcriptome-based Development of AAV Vectors and Promoters

### Materials and Methods

### Library Synthesis

Libraries of AAV with either (a) capsids or (b) promoter/enhancers are engineered to contain unique DNA barcodes. Multiple barcodes are present for each unique construct. Capsid libraries contain capsids with random mutations, semi-random peptide motifs, and random amino acid motifs across surface exposed locations and are based on naturally occurring serotypes, mixtures of naturally occurring serotypes, or synthetic sequences. Enhancer/promoter libraries contain motifs and sequences mined from single cell ATAC-Seq experiments, synthetic sequences, and mutated versions of existing promoters.

To achieve a direct correlation between AAV capsid variants and their barcodes, cap sequences and AAV genomes are encoded on a single plasmid. Libraries are synthesized with mutated cap genes directly upstream of a unique barcode, and the sequences between mutated cap genes and the barcode are cloned between the two (FIG. 1). Variant-barcode pairings are re-confirmed by deep sequencing (high throughput sequencing, such as an ILLUMINA sequencing) before and after packaging.

For example, FIG. 1 depicts the strategy and maps of packaging constructs for single-cell AAV capsid and promoter library screening. Either Cap genes or enhancers, as well as barcodes, are synthesized, cloned into a backbone, and then additional sequence is cloned between the cap gene and barcode, to complete the packaging plasmid.

For AAV capsid libraries, synthesizing libraries in this way allows for both the capsid and genome of a virus to be encoded in the same plasmid. Each HEK293 packaging cell transfected with the plasmid will package a virus containing the barcode designating its unique capsid. Optimal transfection MOIs are determined prior to each packaging to minimize or prevent cross-packaging, by determining the minimal amount of library plasmid DNA required for sufficient packaging (the amount of DNA needed to produce AAV titers of >E+12 vg/mL). Multiple barcodes for each serotype are included ensure that background noise from any potential cross-packaging is reduced. Enhancers are cloned into backbones containing minimal promoters known to drive different levels of expression (such as but not limited to minimal cytomegalovirus (CMV), heat shock protein 68 (HSP68), GATA binding factor 2 (GATA2), and sterol carrier protein (SCP2)), or promoter sequences computationally determined to be related to identified enhancers. Different minimal promoters are identified by an additional 3 base pair tag present in the backbone.

### Single-cell Selection of AAV Capsids and Promoters

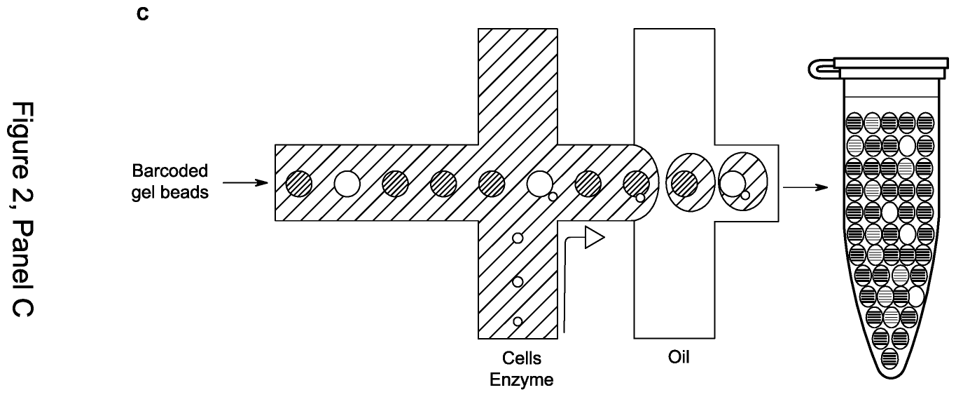

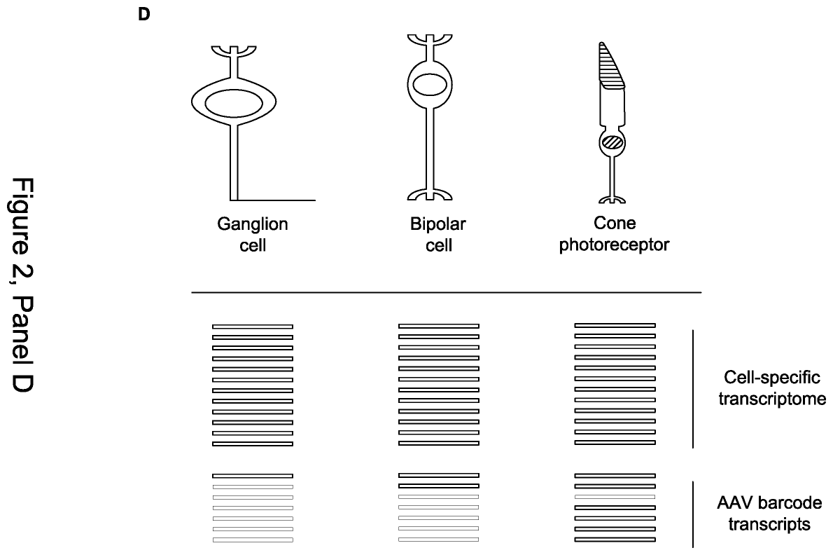

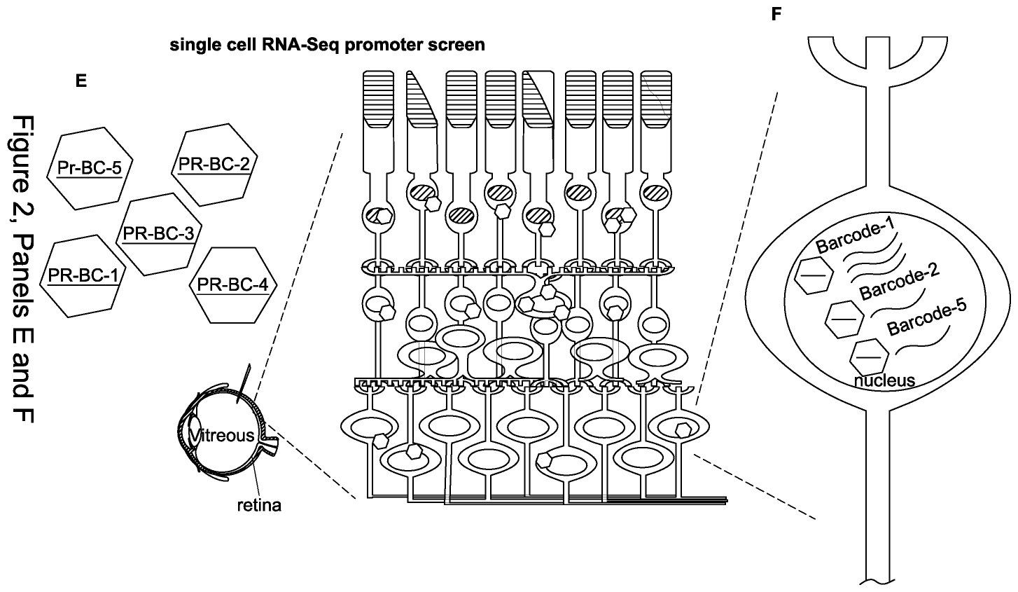

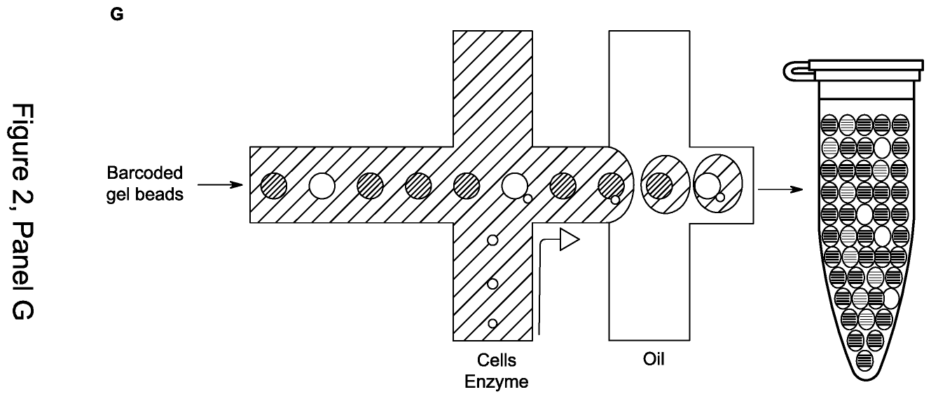

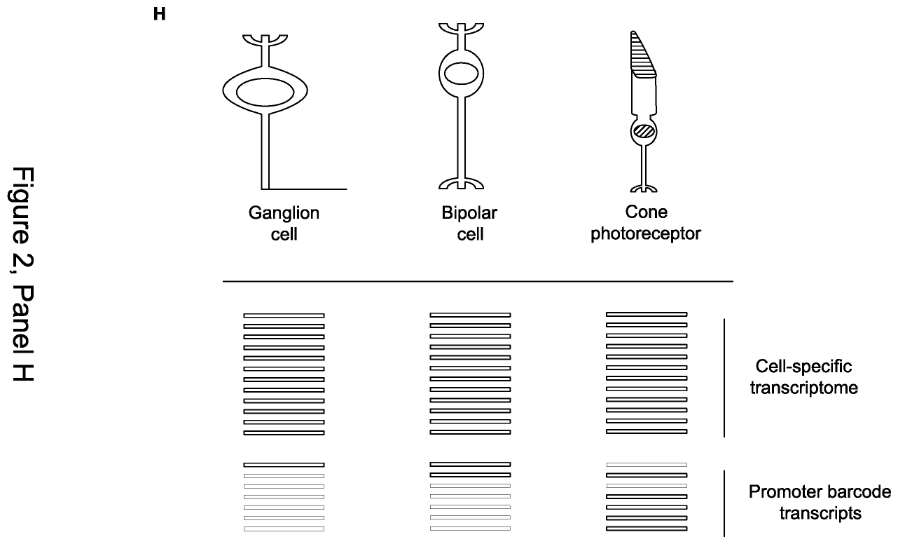

To evaluate the performance of each member of the capsid and promoter libraries, scRNA-Seq is used to identify cell types and to quantify vector and promoter efficiency and specificity, as shown in FIG. 2.

Capsid libraries that contain unique barcodes for each capsid are injected, and vectors infect cells with varying tropisms, efficiencies, and specificities. Single cell suspensions are created from injected tissues, and cells are identified by their transcriptome profile. Simultaneously, capsid performance is quantitatively evaluated by the number of GFP-barcode transcripts recovered from variants. Promoter libraries are packaged into a single variant with broad tropism, and each promoter is paired with a unique barcode. Viruses packaged with enhancer library members infect cells and then, following scRNA-Seq, specificity and efficiency are quantified by counting GFP-barcode transcripts across cell types.

FIG. 2 summarizes an exemplary method provided herein involving single-cell screening of AAV capsids and promoters, using retinal tissue as an example. For Panel A, libraries of barcoded AAVs are injected into tissue. Virus variants from the library infect different cells with different efficiencies. For Panel B, efficient viruses enter cells, traffic to the nucleus, and lead to expression of mRNA. For Panel C, tissue is dissociated into single cells, and mRNA from individual cells is tagged with cell-specific DNA barcodes. For Panel D, the transcriptome profile of individual cells is analyzed to determine cell type, as well as which AAVs have infected the cell, and AAV specificity and efficiency. For Panel E, for enhancer/promoter libraries, the libraries are packaged in a single AAV capsid with broad tropism. For Panel F, different promoters drive varying levels of gene expression in individual cell types. For Panel G, single cell suspensions are created and mRNA from individual cells is tagged with cell-specific DNA barcodes. For Panel H, the transcriptome profile of individual cells is analyzed to identify cell types and determine promoter specificity and efficiency.

### Quantitative Comparison of Subsets of Top-performing Capsids and Promoters

To validate the performance of top candidate capsids and enhancers, a secondary round of evaluation can be performed. The top vectors targeting specific cell types (viruses with greatest level of transgene expression relative the parental serotype, or viruses with greatest level of transgene expression and lowest level of transgene expression in all other cell types relative to the parental serotype) are selected. The most efficient viruses (variants driving most copies of barcode transcripts, normalized by total number of transcripts per cell) and the variants driving most specific and efficient expression (variants driving most copies of on-target barcode transcripts and lowest copies of off-target transcripts) and the variants driving patterns of gene expression most closely matching the wild type pattern of expression of a disease-causing gene, are selected for each cell type or gene. Variants with varying levels of off-target expression are selected for secondary screening to determine an optimal threshold for off-target reads. These will again each be packaged with unique barcodes (such as, but not limited to, GFP-fused barcodes), pooled, injected into tissue, and screened again by scRNA-Seq to determine overall top performing vectors.

Top performing capsids and promoters then are paired and individually validated. The performance of top candidate vectors and promoters is quantified and ranked, and overall top performers for each cell type are identified. Expression profiles are further evaluated in retina and brain by in vivo imaging, histology, qRTPCR, and scRNA-Seq.

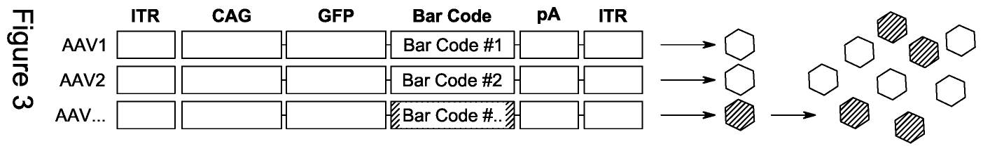

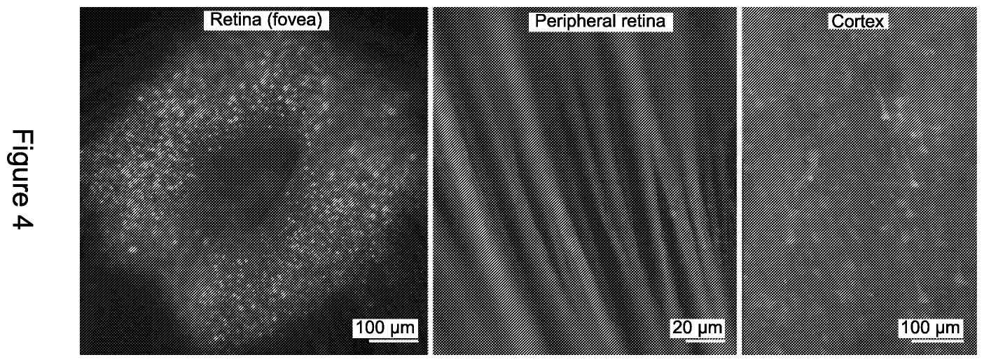

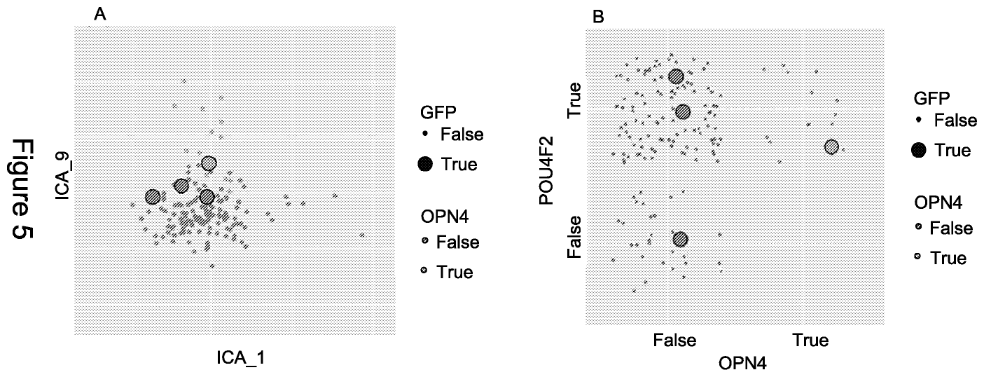

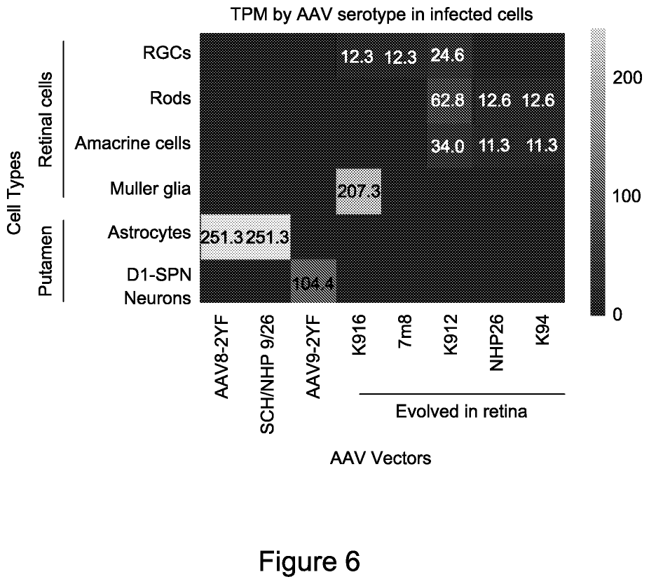

Thus, in accordance with the experiments provided herein, a single cell method of AAV screening described herein was used to evaluate the performance of AAV vectors in different cell types. In retina, intrinsically photosensitive retinal ganglion cells (ipRGCs) were identified as a subset of RGCs, and barcodes were recovered from these cells (FIG. 5). Single cell RNA-sequence analysis of AAV serotypes from retinal and brain cells revealed that, as expected, retina-evolved serotypes performed best in retina, while AAV9 and AAV92YF performed best in putamen neurons (FIG. 6).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Example 2—AAV Libraries

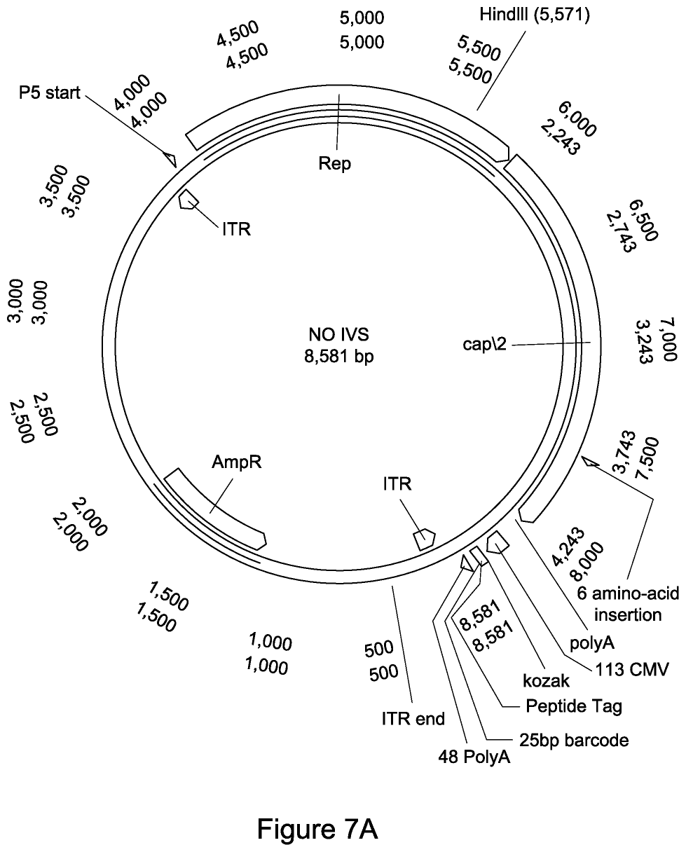

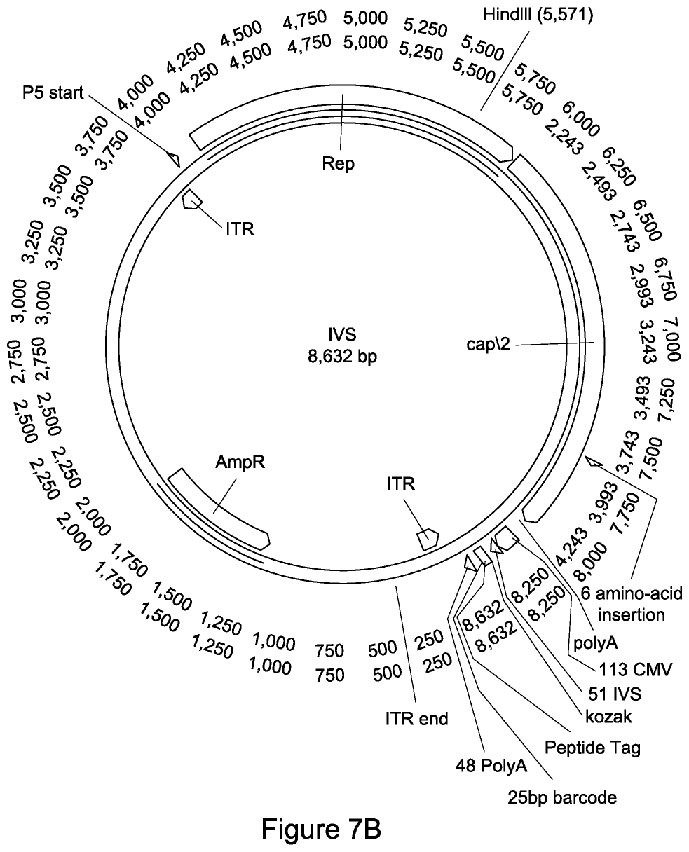

Additional versions of AAV libraries are constructed so that the AAVs use the native conformation of the AAV genome, with an additional small peptide located within the genome (FIGS. 7A and 7B). Briefly, the wild-type AAV genome is retained inside the ITR packaging signals. A small peptide tag, driven by a small ubiquitous promoter (e.g., a miniCMV promoter) and a small minimal pA signal (e.g., a 48 bp polyA signal), is added after the cap open reading frame and between the ITRs (FIG. 7A). This library may contain an intervening sequence (IVS) between the minimal promoter and small peptide sequence (FIG. 7B).

### Example 3—AAV Libraries

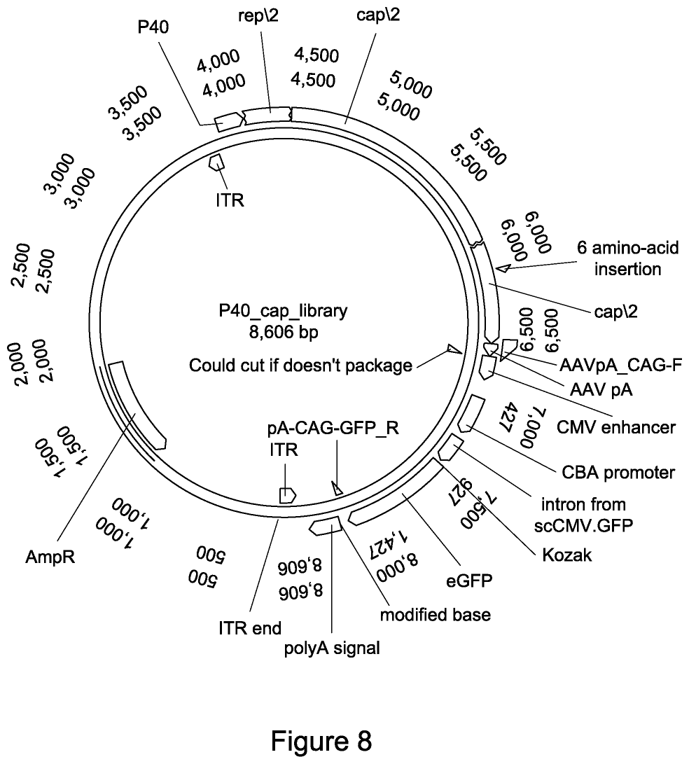

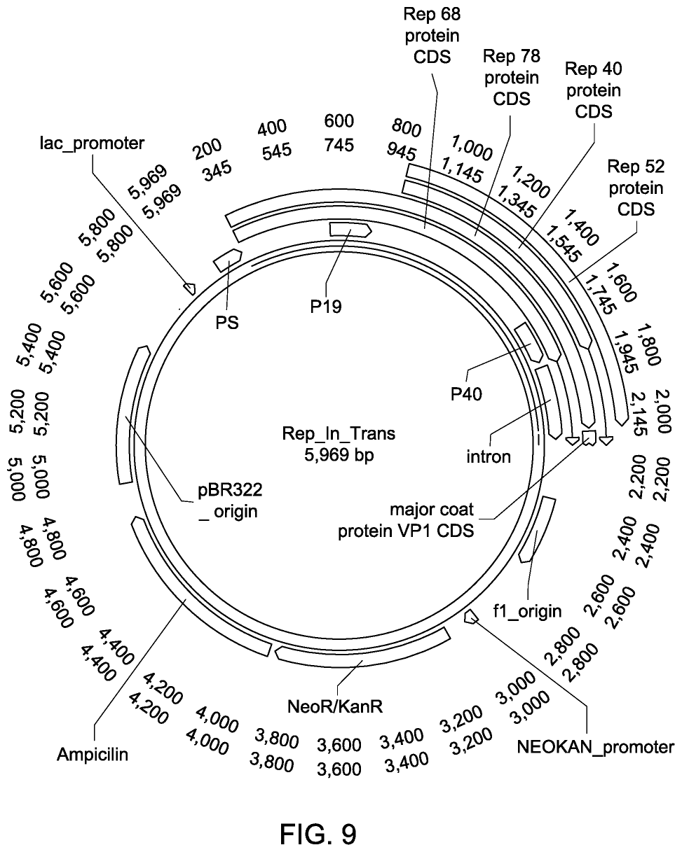

Additional versions of AAV libraries are constructed so that a large strong ubiquitous promoter drives expression of GFP or split GFP (a split GFP can be displayed on the cell surface or retained in the cytoplasm of the cell) and so that the cap gene is packaged within the ITRs (FIG. 8). In these cases, the libraries are created with a rep in trans system (FIG. 9), which has the added benefit of producing replication incompetent libraries for increased safety in animals which may harbor helper virus infections, but allows cap to be driven by endogenous AAV promoters, and cap to be packaged inside the virus, along with a series of barcodes indicating the amino acid insertion in the cap gene. Briefly, these libraries can contain an AAV P40 promoter, an AAV P19+P40 promoter, or a longer version of an AAV P19+P40 promoter. The construct also can contain a promoter such as a ubiquitous CAG promoter that drives expression of a fluorophore like GFP, GFP11 (e.g., split GFP), or GFP11 expressed on the cell surface.

### Example 4—Designing Libraries for Cell-type Specific Promoters using Scatac-seq Data

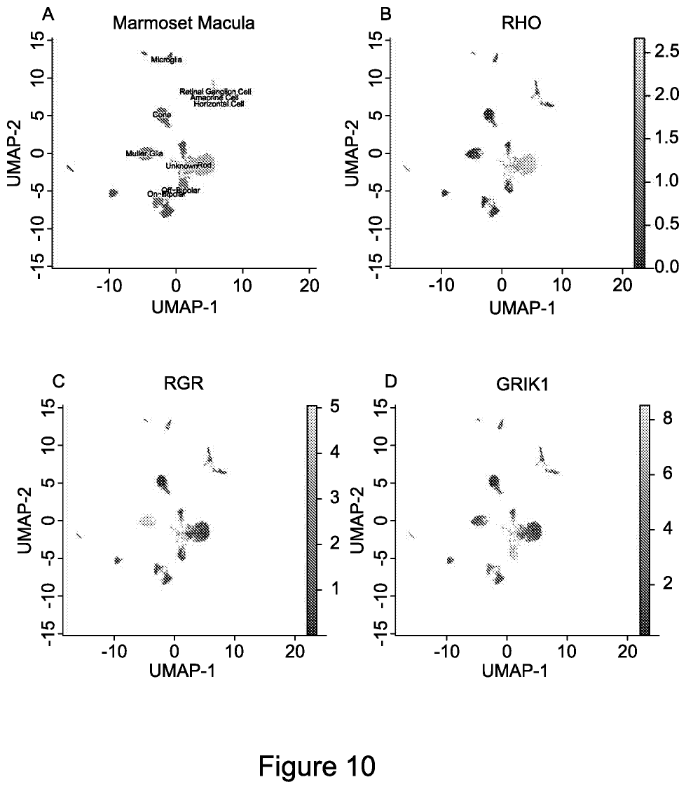

Promoter libraries are built from scATAC data sets, such as those used to cluster single cells as shown in FIG. 10. Single-cell ATACseq is used to determine regions of open chromatin in dissociated retinal cells. The clusters are created from the marmoset macula scATAC-seq data (using SnapATAC) and then integrated with scRNA-seq data from the same sample. Cell types are predicted based on gene expression from the integrated scRNA-seq data (using Seurat). Promoter libraries are constructed by pairing together multiple DNA sequences from specific cell types.

### Example 5—AAV Selection for Disease-specific AAVs

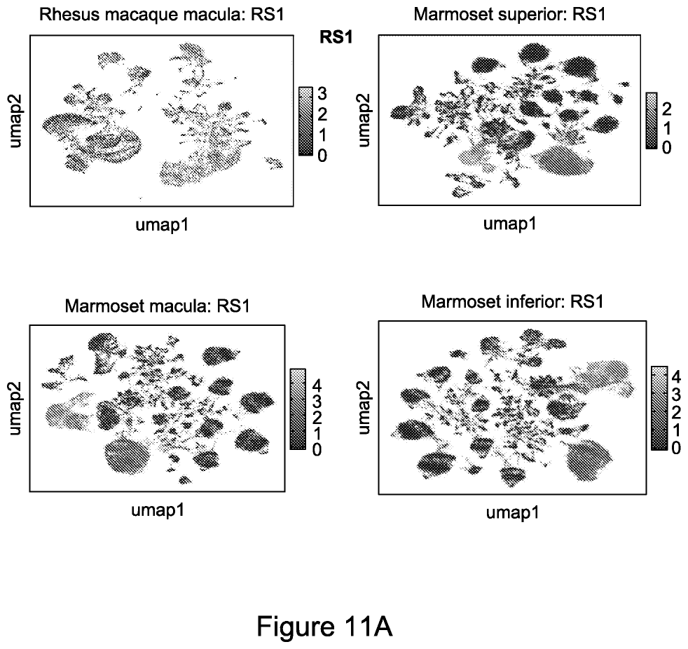

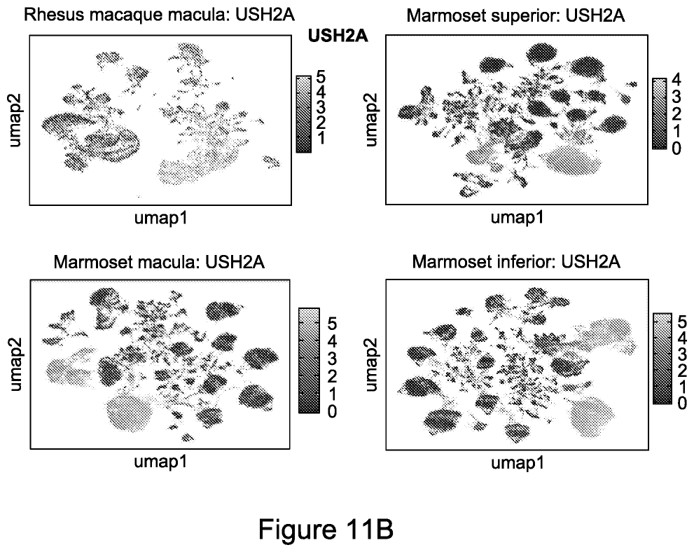

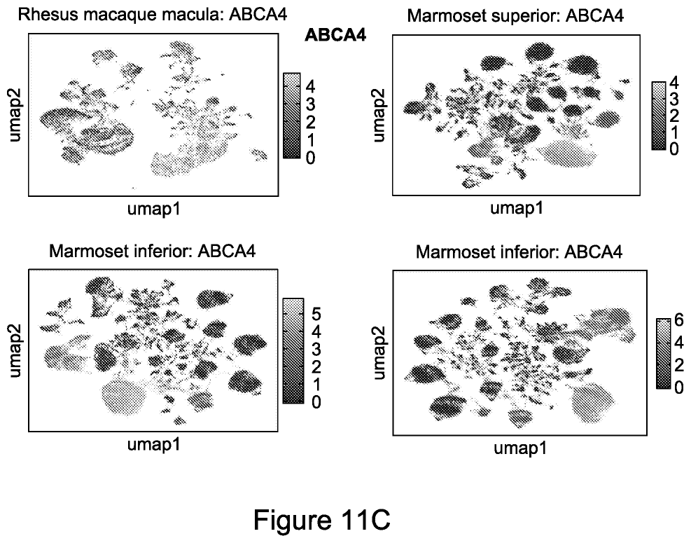

The profile of disease genes can be determined by single-cell RNA-Seq, and then a desired AAV (providing a natural pattern and level of expression of a wildtype copy of a gene) can be determined by matching AAV profiles to disease gene profiles. Disease gene profiles were determined for RS1, USH2A and ABCA4 (FIGS. 11A, 11B, and 11C, respectively).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
(a) creating a library of AAV mutants or promoters, wherein each AAV within the library comprises a unique DNA barcode, or each promoter construct comprises a unique DNA barcode,
(b) packaging of AAV mutants or promoters with a double (for capsid libraries) or triple (for promoter libraries) transfection protocol into a packaging cell line,
(c) delivering the library of AAV mutants into one or more tissues of an animal host, or infecting tissue in culture,
(d) maintaining the library of AAV mutants in vivo or culturing the library of viruses in tissue in culture for a period of time suitable for the AAV vectors within the library of AAV mutants to compete with each other within the one or more tissues of an animal host or cultured tissue into which the library of AAV mutants has been delivered, and
(e) employing single cell or single nucleus microfluidics methodologies to create single cell or single nucleus cDNA libraries from cells within the one or more tissues of the animal host into which the library of AAV mutants has been delivered.

2. The method of claim 1, wherein step (e) employs single cell microfluidics technology.

3. The method of claim 1, wherein step (e) employs single nucleus microfluidics technology.

4-7. (canceled)

8. The method of claim 1, wherein the one or more tissues of an animal host comprises retinal tissue.

9-12. (canceled)

13. The method of claim 1, wherein the animal host is a primate.

14-18. (canceled)

19. The method of claim 1 wherein the delivery of the library of AAV mutants is via injection into the tissue.

20. A method for obtaining an AAV mutant having the ability to infect a desired cell type in vivo and be maintained in vivo within said cell type for at least one week, wherein said method comprises:
(a) introducing a library of AAV mutants into an animal host comprising said cell type, wherein each AAV within said library comprises a unique DNA barcode, and
(b) identifying one or more AAV mutants, based on said barcode for said one or more AAV mutants, as being present in a cell of said cell type, wherein said cell was within said animal host for at least one week after said library was introduced into said animal host.

21. The method of claim 20, wherein said cell type is a central nervous system cell type or peripheral nervous system cell type.

22. The method of claim 20, wherein said cell type is a retinal cell type, a striated muscle cell type, a cardiac muscle cell type, or a smooth muscle cell type.

23. The method of claim 20, wherein said animal host is a primate.

24-28. (canceled)

29. The method of claim 20, wherein said library is introduced into said animal host via injection into tissue comprising said cell type.

30. The method of claim 20, wherein said at least one week is from one week to 12 weeks.

31. A method for obtaining a promotor sequence from a library of AAV viruses, wherein said method comprises:
(a) introducing said library into an animal host comprising a cell type, wherein each AAV within said library comprises a unique promotor sequence configured to drive expression of a fluorescent polypeptide, and
(b) identifying one or more promotor sequences, based on said expression of said fluorescent polypeptide, as being present in a cell of said cell type, wherein said cell was within said animal host for at least one week after said library was introduced into said animal host.

32. The method of claim 21, wherein said cell type is a central nervous system cell type or peripheral nervous system cell type.

33. The method of claim 21, wherein said cell type is a retinal cell type, a striated muscle cell type, a cardiac muscle cell type, or a smooth muscle cell type.

34. The method of claim 31, wherein said animal host is a primate.

35-39. (canceled)

40. The method of claim 31, wherein said library is introduced into said animal host via injection into tissue comprising said cell type.

41. The method of claim 31, wherein said at least one week is from one week to 12 weeks.

42. An isolated nucleic acid comprising nucleic acid encoding an AAV rep polypeptide, nucleic acid encoding an AAV cap polypeptide, and a nucleic acid cassette, wherein said nucleic acid cassette comprises a promotor sequence, nucleic acid encoding a peptide tag, a nucleic acid barcode, and a polyA tail sequence.

43. The isolated nucleic acid of claim 42, wherein said nucleic acid encoding said AAV rep polypeptide, said nucleic acid encoding said AAV cap polypeptide, and said nucleic acid cassette are located between two inverted terminal repeats.

44. The isolated nucleic acid of claim 42, wherein said nucleic acid barcode is between 20 and 30 nucleotides in length.

45. The isolated nucleic acid of claim 42, wherein said isolated nucleic acid is a plasmid.

46. An isolated nucleic acid comprising nucleic acid encoding an AAV cap polypeptide and a nucleic acid cassette, wherein said nucleic acid cassette comprises a promotor sequence, nucleic acid encoding a fluorescent polypeptide, and a polyA tail sequence, and wherein said isolated nucleic acid lacks nucleic acid encoding a full length rep polypeptide.

47. The isolated nucleic acid of claim 46, wherein said nucleic acid encoding said AAV cap polypeptide and said nucleic acid cassette are located between two inverted terminal repeats.

48. The isolated nucleic acid of claim 46, wherein said isolated nucleic acid comprises nucleic acid encoding a rep polypeptide amino acid sequence that is no more than 25 percent, no more than 50 percent, no more than 75 percent, or no more than 85 percent of the amino acid sequence of a full length rep polypeptide.
